(19) United States
(12) Patent Application Publication
(10) Pub. No.: US 2012/0271223 A1
(43) Pub. Date: Oct. 25, 2012

(54) Central Nervous System Drain

(75) Inventor: Rohit Khanna, Daytona Beach, FL (US)

(73) Assignee: NeuroVention, LLC, Daytona Beach, FL (US)

(21) Appl. No.: 13/535,072

(22) Filed: Jun. 27, 2012

Related U.S. Application Data
(62) Division of application No. 12/008,611, filed on Jan. 11, 2008.
Application No. 12/008,611 is a continuation of application No. 11/418,849, filed on May 5, 2006, now Pat. No. 8,123,789.

(51) Int. Cl. A61M 37/00; A61M 1/00

(52) U.S. Cl. 604/22; 604/540; 604/319

(57) ABSTRACT

The invention provides a method and apparatus for maintaining central nervous system drain patency. Ultrasound energy delivered through the drain dissolves the hemorrhage and debris occluding the drain lumen and ports.

### CROSS-REFERENCE TO RELATED APPLICATIONS

The present Utility patent application is a division of U.S. non-provisional application for patent Ser. No. 12/008,611 entitled “Central nervous system ultrasonic drain”, filed on Jan. 11, 2008, which is a continuation of U.S. non-provisional application Ser. No. 11/418,849 filed on May 5, 2006, now U.S. Pat. No. 8,123,789. The contents of this related application are incorporated herein by reference for all purposes to the extent that such subject matter is not inconsistent herewith or limiting hereof.

### BACKGROUND OF THE INVENTION

Central nervous system disease frequently requires placement of burr holes or craniotomies for exposure of the brain and intracranial contents for various intracranial pathologies including tumors, head injuries, vascular malformations, aneurysms, infections, hemorrhages, strokes, and brain swelling. A craniotomy involves creation of burr holes and removal of a portion of the skull (bone flap) with subsequent exposure and treatment of the underlying pathology. In regards to spine pathology, the usual exposure involves complete or partial removal of the lamina, disc or vertebral body. Percutaneous spinal exposure through the interlaminar or foraminal space can also be achieved. These procedures routinely also involve placement of a surgical drain to reduce pressure from either fluid or hemorrhage accumulation. Surgical drain obstruction is a very common and debilitating problem in these patients.

A ventriculostomy or also referred to as an external ventricular drain is routinely placed to monitor and treat elevated intracranial pressure in patients with severe traumatic brain injuries, non-traumatic cerebral or intraventricular hemorrhages, hydrocephalus, and cerebral swelling. Unfortunately, acute hemorrhage turns into a blood clot within a few minutes and therefore, does not drain out through a tube until it dissolves. This natural blood clot dissolution process can take several days to weeks. A ventriculostomy not infrequently gets obstructed from either blood clots or debris which, in turn also foster infectious complications.

Consequently, there remains a great margin for improvement, particularly with treatment options providing for a faster, less invasive, and a low complication approach for central nervous system drain obstruction.

Several strategies to treat central nervous system drain obstruction through the use of ultrasound have been described in U.S. patent application Ser. No. 12/008,611, the entirety of which are hereby incorporated by reference herein. The interaction between ultrasound and a thrombolytic agent has been shown to assist in the break-down or dissolution of a blood clot, as compared with the use of the thrombolytic agent alone.

### SUMMARY OF THE INVENTION

The present invention describes a central nervous system drain capable of maintaining lumen patency. Ultrasonic energy is used to hemolyse and dissolve blood clots and/or debris occluding the drain lumen and ports. The clot hemolysis can be facilitated with the use of thrombolytic, hemolytic, antiplatelet, anchor anticoagulant agents also delivered through the drain. The dissolved clot is then drained through the drain either via dependent gravity drainage or a suction apparatus. Placement of the drain utilizes a well versed “burr hole” technique commonly practiced in the field of neurosurgery for placement of a ventriculostomy drain and cerebral pressure monitoring devices. Typically, a small skin incision is made in the head using standard external landmarks. A small hole in the skull is then created with the use of a drill and subsequently the drain is then placed into the brain or subdural space. A precise placement of the drain can be facilitated with the use of stereotactic techniques if needed. The drain can also be placed following a craniotomy or laminectomy.

Ultrasonic energy focused upon a blood clot causes it to break apart and dissolve. This process termed thrombolysis liquefies the clot and allows subsequent drainage through the drain. Depending on the frequency of the ultrasonic energy used, the ultrasound effect is carried through by means of mechanical action, heat, or cavitation. The lower frequency acoustical waves, usually below 50 KHz, dissolve a blood clot by cavitation and frequencies above 500 KHz take affect more so by generating heat. These waves can be focused to produce a therapeutic effect up to 10 cm or more from the transducer.

Ultrasonic energy can be transmitted either through an external transducer connected to a conductor in the drain or through a transducer located in the drain. An ultrasonic transducer converts electrical energy into ultrasonic energy through a piezoelectric ceramic or similar element. The ultrasound conductors can be embedded in the drain wall or lumen and can comprise of wires or any other shape suitable for ultrasound conduction and/or amplification. Alternatively, the ultrasound transducers can be embedded in the drain wall or lumen with electrical wires connecting the transducers to an external electrical source. The ultrasonic member in the drain lumen can either be permanent or removable.

The ultrasonic frequency waves can also be generated continuously or in a pulsed format. Use of continuous waves allows clot dissolution in a shorter time period but also generates more heat. Pulsed waves prevent heat build-up and reduce the risk of cavitation in the target tissue, but may also take affect over a longer period of time. For example, at frequencies in the range from 50 to 150 MHz, dissolution only occurs in close proximity to the face of the transducer with the actual distance depending upon the elastic and acoustical properties of the propagating medium. Adverse rises in temperature are also prevented, preferably by selecting a pulsed mode of operation, such that coagulation of tissue and other disadvantageous side-effects accompanying adverse temperature rises can be avoided. Applying ultra-high frequency energy 50 MHz to 100 GHz to the hemorrhage in pulses, rather than as a continuous wave, may actually reduce the time required to dissolve tissue structures; however continuous wave application is also effective. In pulsed mode operation, for example in pulses of about 10 to about 100 wavelengths in duration, substantially higher wave amplitudes, but lower energy densities, can be applied to the hemorrhage with the assurance that any high-frequency vibratory mode imparted to the hemorrhage by the acoustical waves will also be absorbed within the localized area of the target tissue.

Whereas relatively low frequency ultrasonic devices break apart the hemorrhage by mechanical impact or cutting action, a radiated propagating wave of high frequency ultrasonic energy, preferably in short pulses, dissolves blood clots into its cellular/sub cellular components in a highly controlled and localized manner.

In some instances, cooling may be needed to avoid the adverse effects of temperature rises by ultrasound energy use. Several methodologies and cooling catheters have been described in U.S. Pat. No. 8,123,789 to counteract this heating effect, the entirety of which are hereby incorporated by reference herein.

Ultrasound frequency in the 100 MHz range can be used to dissolve blood clots in a very localized region within I mm of the transducer without deleteriously affecting the surrounding brain. By contrast, acoustical waves at 1 MHz travel about 3 cm before attenuation reduces its power by one half.

Similarly, wavelength helps to determine the type of destructive forces that operate in target material and the size of the particles generated. When the wavelength of sound is relatively long, cavitation and/or gross mechanical motion produce the blood clot break-up. Such a situation certainly exists if the frequency of the sound is around 40 kHz or below. When, however, the wavelength of sound is very much smaller, as it is at 100 MHz, the mechanical energy associated with the propagating sound wave breaks down the blood clot into cellular or sub cellular components. The depth of material breakdown as measured from the surface of the material to be treated is frequency dependent and the blood clot can be dissolved to a microscopic level by selecting the appropriate frequency. It has also been shown that a 100 MHz ultrasound frequency can dissolve blood clots by using a pulsed sequence without cavitation or heat generation using mainly a mechanical breakdown effect.

The process by which thrombolysis is affected by use of ultrasound in conjunction with a thrombolytic agent can vary according to the frequency, power, and type of ultrasonic energy applied, as well as the type and dosage of the thrombolytic agent. The application of ultrasound has been shown to cause reversible changes to the fibrin structure within the thrombus, increased fluid dispersion into the thrombus, and facilitated enzyme kinetics. These mechanical effects beneficially enhance the rate of dissolution of thrombi. In addition, ultrasound induced cavitational disruption and heating/streaming effects can also assist in the breakdown and dissolution of thrombi.

The thrombolytic agent can comprise a drug known to have a thrombolytic effect, such as streptokinase, urokinase, prourokinase, ancrod, tissue plasminogen activators (alteplase, anistreplase, tenecteplase, reteplase, duteplase. Alternatively (or in combination), the thrombolytic agent can comprise an anticoagulant, such as heparin or warfarin; or an antiplatelet drug, such as a GP IIb IIIa, aspirin, ticlopidine, clopidogrel, dipyridamole; or a fibrinolytic drug such as aspirin. Alternatively the thrombolytic agent can be incorporated into micro bubbles, which can be ultrasonically activated after direct infusion into the blood clot through a catheter.

It may be possible to reduce the typical dose of thrombolytic agent when ultrasonic energy is also applied. It also may be possible to use a less expensive or a less potent thrombolytic agent when ultrasonic energy is applied. The ability to reduce the dosage of thrombolytic agent, or to otherwise reduce the expense of thrombolytic agent, or to reduce the potency of thrombolytic agent, when ultrasound is also applied, can lead to additional benefits. such as decreased complication rate, and an increased patient population eligible for the treatment.

Drains capable of delivering ultrasonic energy can be placed directly into the hemorrhage inside the skull, brain, or spine and facilitate blood clot dissolution and drainage. In some embodiments of the drainage catheters, ultrasonic energy generated outside the drain is transmitted through conductors in the drain wall or lumen. In other embodiments of the drainage catheters, ultrasonic energy is generated by transducers placed within the drain.

Placement of a subdural drain following either a burr hole placement or craniotomy is a very common methodology practiced in neurosurgery. This drain is very prone to obstruction from the hemorrhage and not infrequently requiring further surgery to evacuate the residual or recurrent hemorrhage development. As described in the current methodology, a drain equipped with delivering ultrasonic energy to the lumen will also dissolve any obstruction from blood clots or debris in the lumen and significantly reduce this complication by maintaining drain patency.

### BRIEF DESCRIPTION OF THE DRAWINGS

FIG. 1 is a schematic view of the ultrasonic drain in the brain.

FIG. 2 is a cross-sectional longitudinal view of one embodiment of the drain.

FIG. 3 is a cross-sectional longitudinal view of another embodiment of the drain.

FIG. 4 is a cross-sectional transverse view of the drain taken along line A in FIG. 2.

FIG. 5 is a cross-sectional view of the drain taken along line B in FIG. 3.

FIG. 6 is a cross-sectional side view of another embodiment of the drain.

FIG. 7 is another cross-sectional side view of another embodiment of the drain shown in FIG. 6 with the removable Ultrasound transducer in the lumen.

FIG. 8 is a cross-sectional view of the drain taken along line A in FIG. 6.

FIG. 9 is a cross-sectional view of the drain taken along line A in FIG. 6.

FIG. 10 is a cross-sectional side view of another embodiment of the drain.

FIG. 11 is a cross-sectional side view of another embodiment of the drain.

FIG. 12 is a cross-sectional view of the drain taken along line A in FIG. 11.

FIG. 13 is a cross-sectional view of the drain taken along line B in FIG. 11.

FIG. 14 is a cross-sectional side view of another embodiment of the drain.

FIG. 15 is a cross-sectional side view of another embodiment. of the drain.

FIG. 16 is a cross-sectional view of the drain taken along line B in FIG. 14.

FIG. 17 is a cross-sectional view of the drain taken along line A in FIG. 14.

FIG. 18 is a cross-sectional side view of another embodiment of the drain.

FIG. 19 is a cross-sectional side view of another embodiment of the drain.

FIG. 20 is a cross-sectional view of the drain taken along line A in FIG. 18.

FIG. 21 is a cross-sectional view of the drain taken along line A in FIG. 19.

FIG. 22 is a cross-sectional view of the drain taken along line B in FIG. 19.

FIG. 23 is a cross-sectional side view of another embodiment of the drain.

FIG. 24 is a cross-sectional side view of another embodiment of the drain.

FIG. 25 is a cross-sectional side view of another embodiment of the drain.

FIG. 26 is a cross-sectional view of the drain taken along line A in FIG. 24.

FIG. 27 is a cross-sectional side view of another embodiment of the drain.

FIG. 28 is a cross-sectional side view of another embodiment of the drain.

FIG. 29 is a cross-sectional view of the drain taken along line A in FIGS. 27 & 28.

FIG. 30 is a side view of another embodiment of the drain.

FIG. 31 is a side view of another embodiment of the drain with the ultrasonic energy generator.

FIG. 32 is a cross-sectional view of another embodiment of the drain.

FIG. 33 is a side view of one embodiment of the ultrasound stylet.

FIG. 34 is a side view of another embodiment of the ultrasound stylet.

FIG. 35 is a side view of the ultrasound energy generator.

FIG. 36 is a schematic side view of another embodiment of the drain.

FIG. 37 is a cross-sectional view of the drain shown in FIG. 36.

FIG. 38 is a cross-sectional side view of another embodiment of the drain with the removable stylet.

FIG. 39 is a side view of another embodiment of the ultrasound stylet.

FIG. 40 is a side view of another embodiment of the ultrasound stylet.

FIG. 41 is a schematic side view of another embodiment of the drain.

FIG. 42 is a cross-sectional view of the drain shown in FIG. 41.

FIG. 43 is a schematic side view of another embodiment of the drain.

FIG. 44 is a cross-sectional view of the drain shown in FIG. 43.

FIG. 45 is a schematic side view of another embodiment of the drain.

FIG. 46 is a cross-sectional view of the drain shown in FIG. 45.

FIG. 47 is a schematic side view of another embodiment of the drain.

FIG. 48 is a cross-sectional view of the drain shown in FIG. 47.

### DETAILED DESCRIPTION OF THE PREFERRED EMBODIMENTS

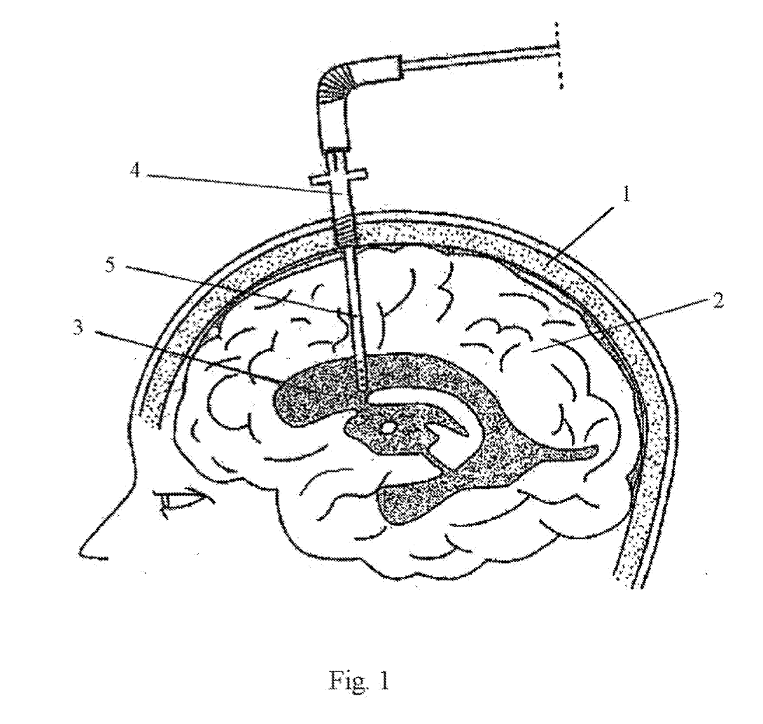

In one embodiment of the central nervous system drain 5 as shown in FIG. 1 can be placed inside the brain 2 or ventricle 3 or the subdural or epidural space. This drain can be placed using the standard landmarks or can be precisely placed with stereotactic guidance or use of an endoscope. A bolt 4 can also be used to secure the catheter through the skull 1 but is not necessary. The drain is placed either through a small drill hole created in the skull or after a craniotomy or burr hole placement.

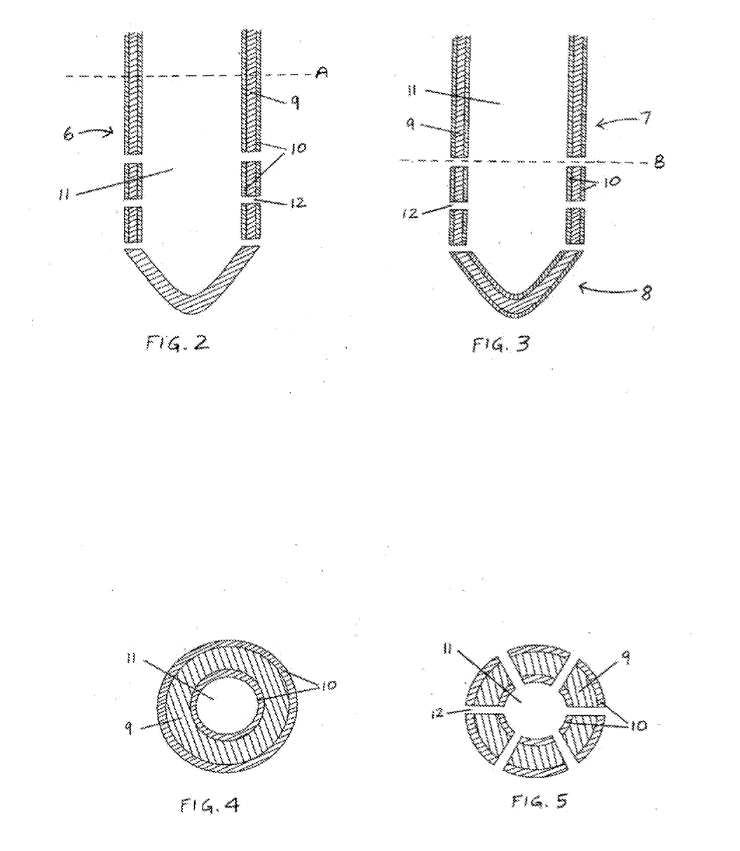

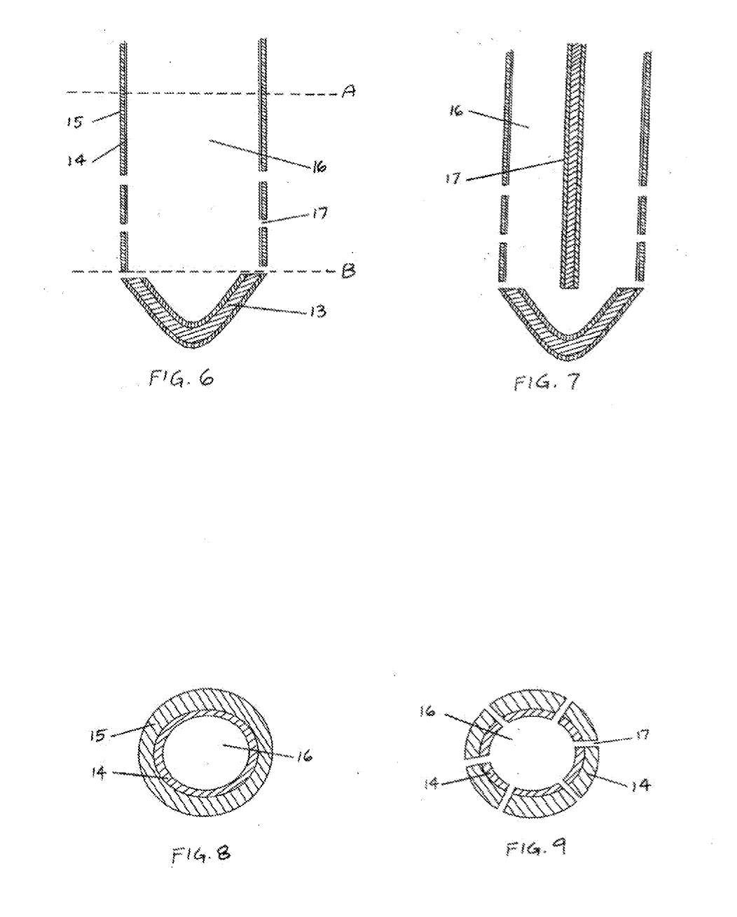

FIGS. 2-5 illustrate another embodiment of the ultrasonic drain. The distal drain wall 6 as seen in FIG. 2 or the wall 7 and tip 8 as seen in FIG. 3 contain the ultrasound transducer with a piezoelectric crystal 9 surrounded by electrodes 10. The drain contains a lumen 11 with ports 12 at the distal ends that communicate with the external environment. When the drain is placed directly into the blood clot, the ultrasonic energy dissolves the clot inside and outside the drain lumen, which can be further facilitated if needed by infusing a hemolytic or thrombolytic or antiplatelet agent through the lumen and then draining the liquefied blood through the same lumen. Since the lumen communicates with the brain, it can also be used to monitor the intracranial pressure.

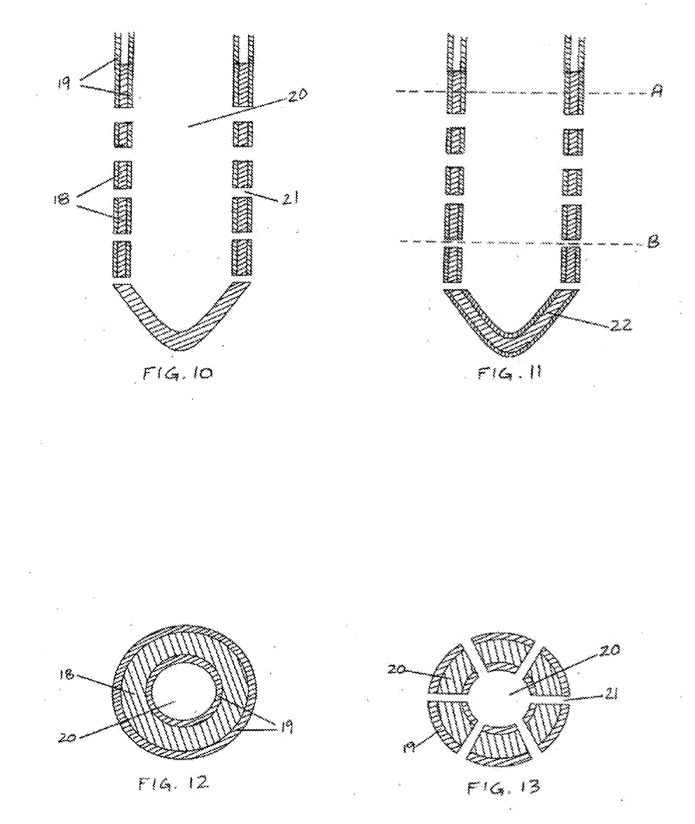

FIGS. 6-9 illustrate an ultrasonic drain with the transducer 13 at the distal tip. The ultrasound transducer electrodes 14 are embedded in the drain wall 15. The drain contains a lumen 16 with ports 17 at the distal end that communicate with the outside environment. As shown in FIG. 7, the lumen 16 can also contain an ultrasound transducer 17 which is removable.

FIGS. 10-13 illustrate an ultrasonic drain with the distal end comprising of a plurality of ultrasound transducers 18 connected to a signal generator at the proximal end through an electrical conductor 19. The drain also has a longitudinal lumen 20 with portals 21 at the distal end. The ultrasound transducers also having a plurality of resonant frequencies and can receive a multi-frequency driving signal to the plurality of ultrasound transducers. In another embodiment, the drain tip 22 as shown in FIG. 11 also contains an ultrasound transducer.

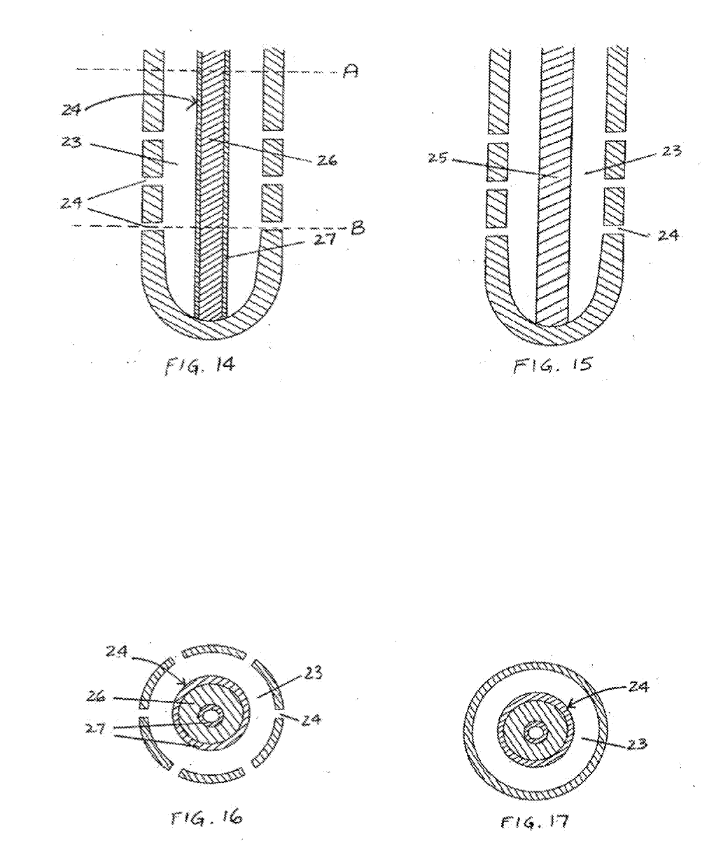

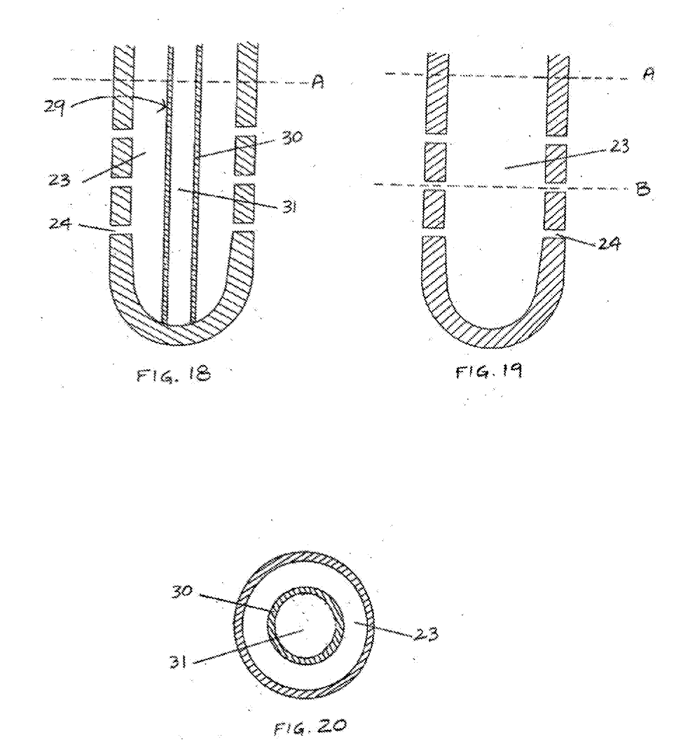

In another embodiment of the ultrasonic drain as illustrated in FIGS. 14-22, the drain contains a lumen 23 which communicates with the outside environment through ports 24. The lumen 23 is also capable of incorporating an ultrasound transducer 24. or conductor 25 which is removable. FIGS. 14, 16, &17 illustrate a drain with an ultrasound transducer 24 in the lumen 23. The transducer consists of a piezoelectric crystal 26 surrounded by electrodes 27. The ultrasound transducer 24 can be inserted or removed as needed for thrombolysis. FIG. 15 illustrates a drain with an ultrasound conductor 25 in the lumen 23. The conductor 28 typically is comprised of a metal that transmits ultrasound energy from a generating source at the proximal end of the drain.

FIGS. 18 & 20 illustrate the drain with an ultrasound conductor 29 in the lumen 23. The conductor 29 has a wall 30 and a lumen 31 filled with a fluid or gel that propagates ultrasonic waves through the catheter from a generating source connected to the proximal end of the drain.

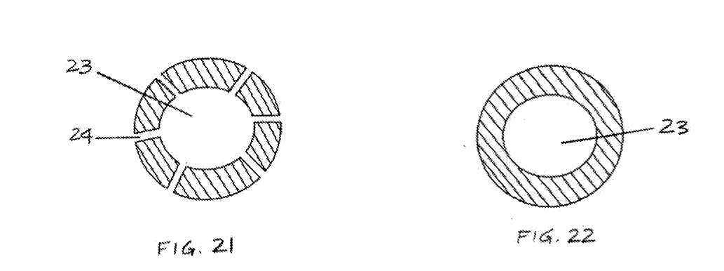

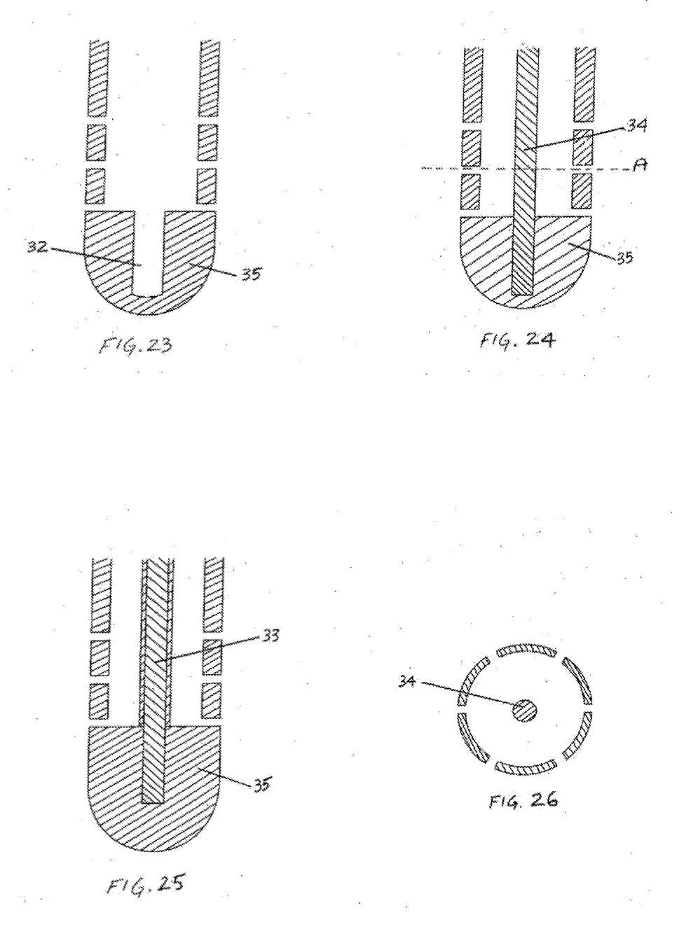

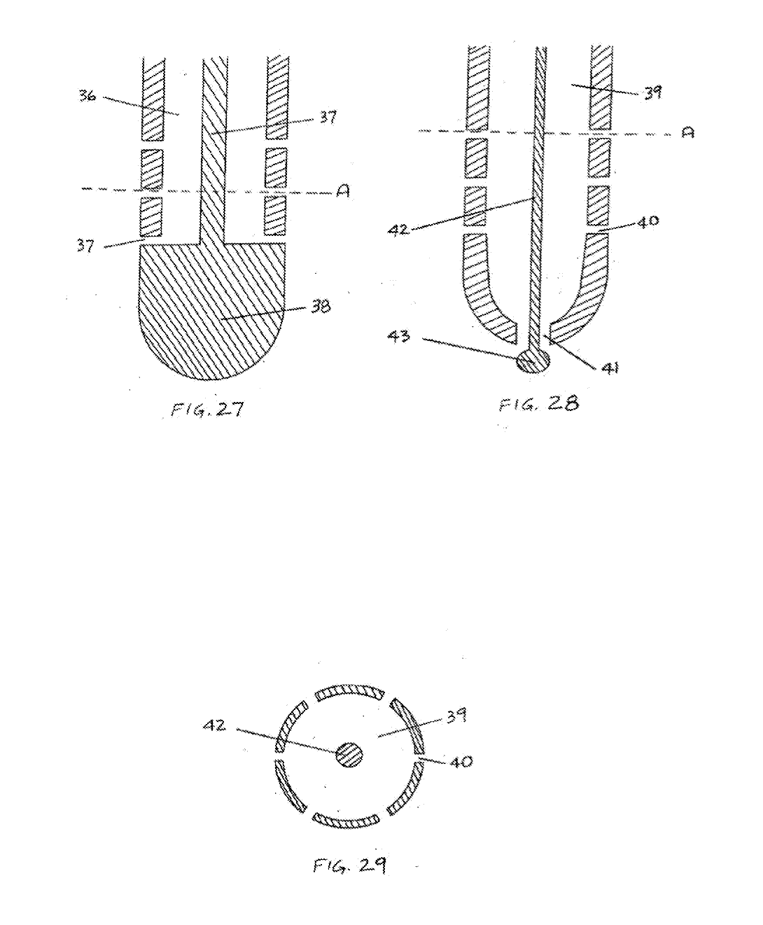

FIGS. 19, 21, &22 illustrate the drain with the transducers removed from the lumen 23.

FIGS. 23-26 illustrate another embodiment of the drain with an anchor 32 at the distal end for the removable ultrasound transducer 33 or conductor 34. This anchor can also serve as an amplifier 35 for the ultrasound energy. FIG. 23 illustrates the drain with the ultrasound transducer removed.

FIG. 27 illustrates another embodiment of the drain with a lumen 36 and ports 37 at the distal end. The lumen 36 contains an ultrasound conductor 37 attached to an amplifier 38 at the tip. Ultrasonic energy is generated from an outside source and transmitted through the conductor and is further amplified by the amplifier at the catheter distal end. FIGS. 28 & 29 illustrate another embodiment of the catheter with a lumen 39 and ports 40 at the distal end and an opening 41 at the tip. The lumen 39 contains an ultrasound conductor 42. The conductor 42 has an enlarged distal end 43 that can extend outside the drain lumen 39 through the opening 41. The enlarged distal conductor end amplifies the ultrasound energy as well as facilitates blood clot hemolysis extending outside the drain tip.

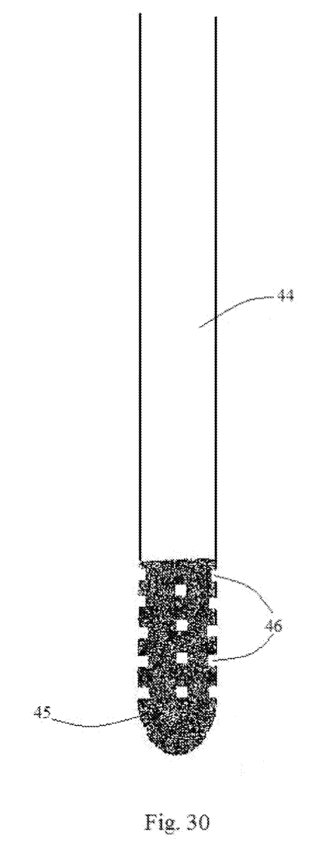

FIG. 30 illustrates the ultrasonic drain best suited for placement in the ventricle. Similar to a ventriculostomy, the drain is circular in shape with multiple perforations at the distal end. It can also contain external markers to indicate the depth of the drain placement either in 1 cm or 5 cm increments. The drain 44 has a distal ultrasound component 45 with multiple ports 46 that connect to the lumen inside the drain. The ultrasound component 45 can comprise of either a transducer with drainage holes or a conductor. The ultrasound transducer is connected to an external electrical source through a wire embedded in the catheter 44 wall. The wires can also be coated for insulation. Alternatively, the ultrasound conductor is connected to an external transducer through one or more wires either embedded in the catheter wall or linked to conductors in the lumen. The conductor(s) in the lumen can be removable and placed when desired for a specific time period ranging from minutes to several days. The drain may also include temperature and pressure sensors. In other embodiments, the ultrasound conductor can also serve as a temperature sensor.

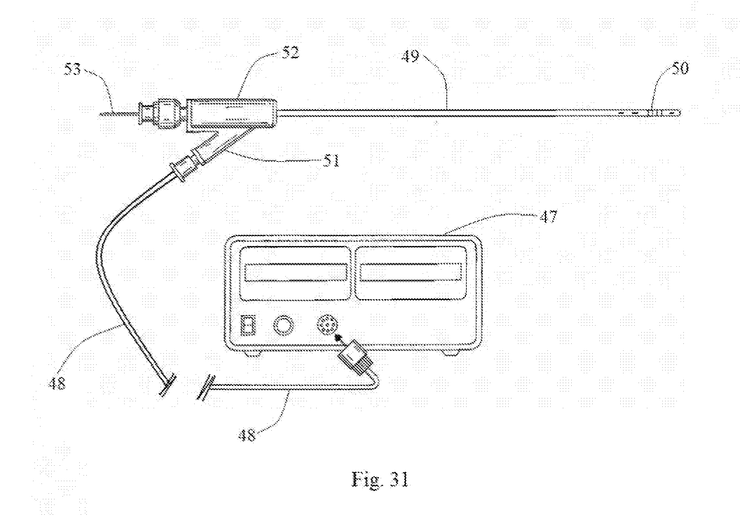

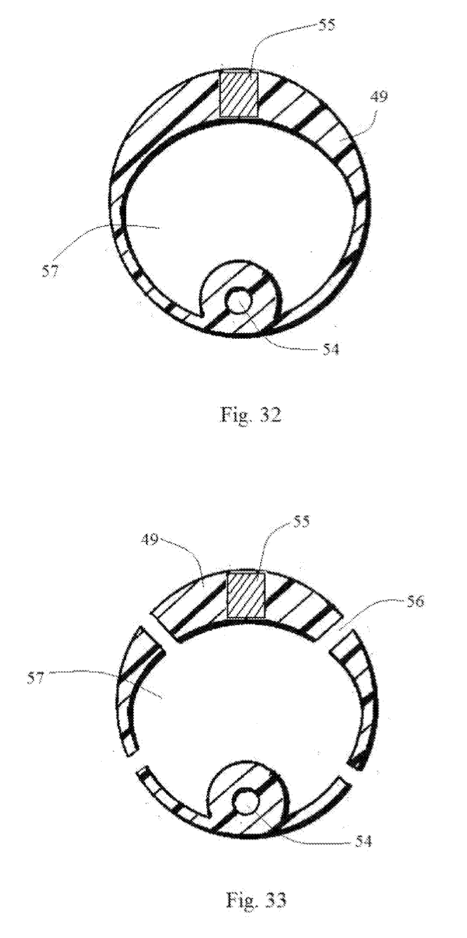

FIG. 31 illustrates an ultrasonic drain 49 with a distal component 50 comprising of drainage ports and an ultrasound component. The proximal drain portion 51 connects the ultrasound component to an external energy source 47 through the connector 48. The external energy source 47 can either comprise an electrical source which transmits electrical energy through the connecting wire 48 into the distal drain end 50 ultrasound component transducers. Alternatively, the external energy source 47 can comprise an ultrasound transducer that is connected to the distal drain end 50 ultrasound component conductors. The drain also comprises a proximal portion 52 that connects the drain lumen to a drainage bag. The drainage proximal portion 52 can also be connected to a vacuum negative pressure device or bag to facilitate drainage. A stylet 53 can also be placed inside the drain 49 lumen to assist in the placement of the drain inside the head or spine. The stylet provides for drain stiffness to target the exact placement location. The stylet or the drain can also be registered with markers for camera sensors for navigational purpose. This allows for stereotactic placement of the drain through image guidance. Alternatively, the drains can also contain or be embedded with radio-opaque markers to visualize location on x-rays or fluoroscopy. The external energy source 47 can be adjusted to provide either continuous or pulsed mode of operation. The pulse repetition rate, duty cycle, average power, and duration can vary and be adjusted as necessary.

In an alternative embodiment, the ultrasonic drain can also contain two lumens, one for drainage and the other for delivery of a hemorrhage lysis agent. FIGS. 32 & 33 illustrate an embodiment of this drain. The drain wall 49 at the distal end of the drain is embedded with one or more ultrasound transducers 55. The lumen 57 is used for drainage and connects to the external environment through ports 56 at the distal portion of the drain. The lumen 54 is used for infusion or injection of a hemorrhage lysis agent.

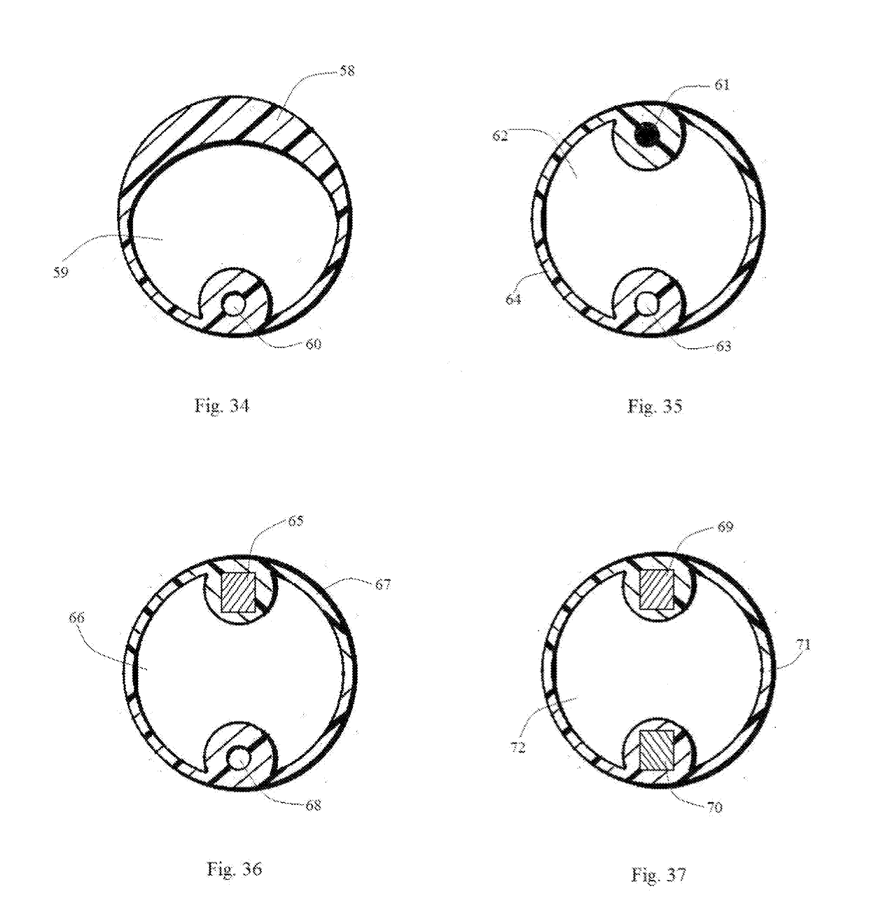

FIG. 34 illustrates another embodiment of the double lumen ultrasound drain with drainage lumen 59, drug infusion lumen 60, and an ultrasound conduction wall 58. FIG. 35 illustrates another embodiment of the drain, with a drainage lumen 62, drug infusion lumen 63, and an ultrasound conductor 61 embedded in the drain wall 64. In another embodiment as shown in FIG. 36, the drain comprises of a drainage lumen, 66, drug infusion lumen 68, and ultrasound transducer 65 secured in an epoxy housing attached to the wall 67. FIG. 37 illustrates another embodiment of the drain with a drainage lumen 72 and ultrasound transducers 69 and 70 at the distal catheter wall 71 each in their own housing unit. The ultrasound transducers can be spaced apart circumferentially and longitudinally at a selected angle and distance from each other so as to provide a uniform delivery of ultrasonic energy within a desired location.

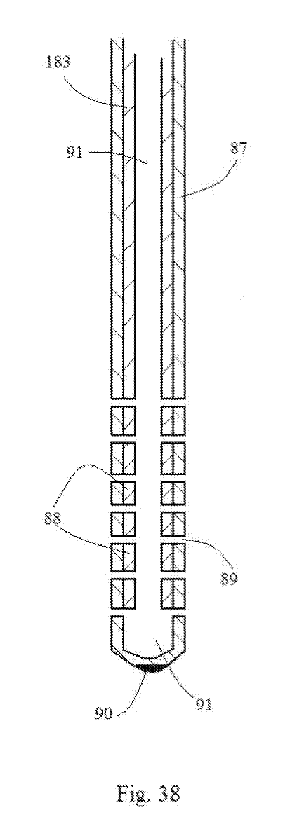

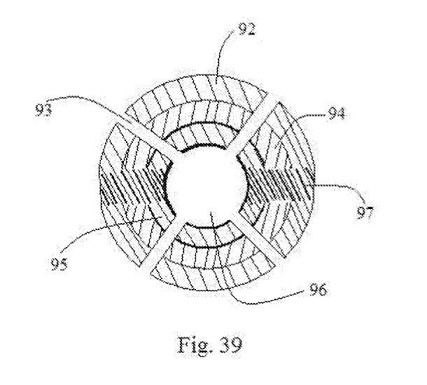

In another embodiment, as shown in FIGS. 38, the drain comprises of a wall 87 and a distal end with an ultrasound transducer 88 and electrical wires 183. The drain comprises drainage holes 89 connecting the lumen 91 to the outside environment. The distal end also comprises of one or more temperature sensors 90. FIG. 39 illustrates a drain with ultrasound transducers 97 embedded in the catheter wall. The catheter wall comprises an outer layer 92 and inner layer 95 for insulation of the electrical wires 94. The distal end of the drain also comprises of ports 93 for drainage into the lumen 96.

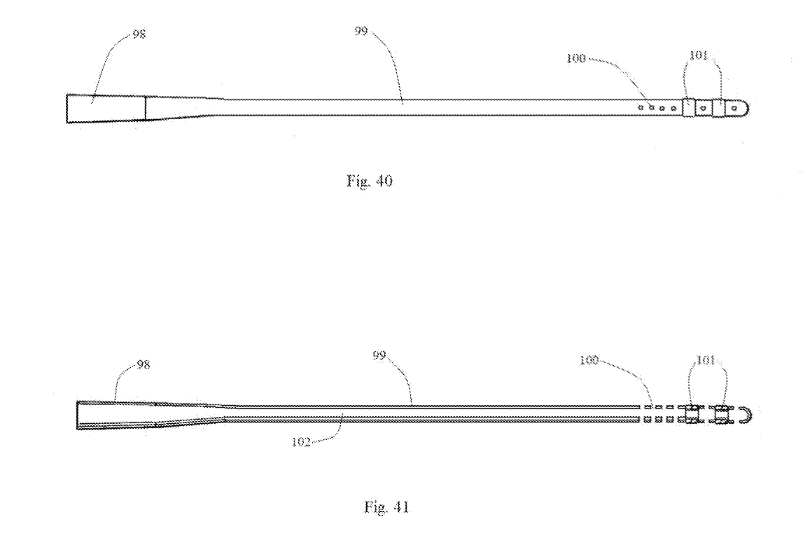

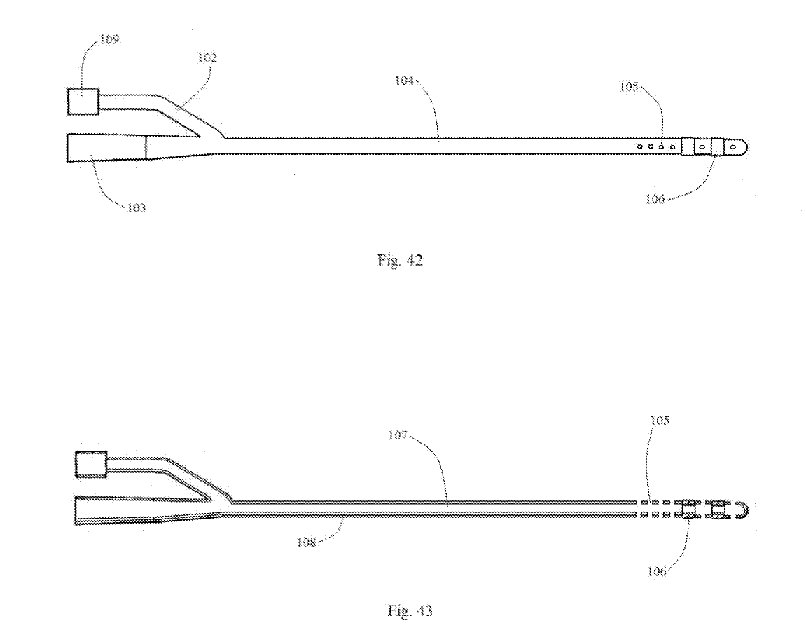

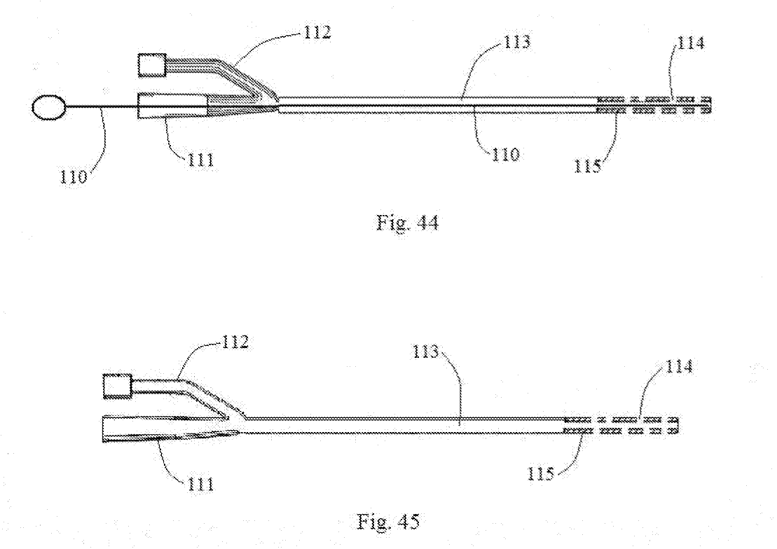

In an exemplary embodiment as shown in FIGS. 40 & 41, the ultrasound drain 99 comprises a proximal portion 98 that connects to a drainage bag and an external source of electrical energy for activation of the ultrasound transducers 101 located at the distal drain end. The drain 99 also comprises of a lumen 102 with drainage holes 100 at the distal end in between the transducers 101. Although, in this illustration two transducers 101 are shown, the number of transducers can vary from one to 5 or higher. In another embodiment, as shown in FIGS. 42 & 43, the drain 104 comprises of a forked proximal end 102 and 103. The end 103 connects the lumen 107 to an external drainage bag or suction device and the end 102 connects to an external energy source for the transducers 106. The end 102 can also comprise of a connector 109 to secure the end to the electrical cable connecting to the external energy source. The catheter 104 distal end is placed inside the central nervous system and comprises of drainage ports 105 that connect to the lumen 107. The distal end also comprises of ultrasound transducers 106 connected to the external energy source through one or more wires 108. FIGS. 44 & 45, illustrate another embodiment of the drain with the distal portion of the catheter 115 comprising of an ultrasound transducer. The distal end transducer has drainage holes 114 and a hollow center drainage lumen 113. The proximal portion 111 of the drain connects the lumen to an external drainage bag. A stylet 110 is used to place the drain inside the central nervous system. The proximal portion 112 connects the distal hollow transducer 115 to an external energy source with electrical wires which can be insulated in the catheter wall or lumen.

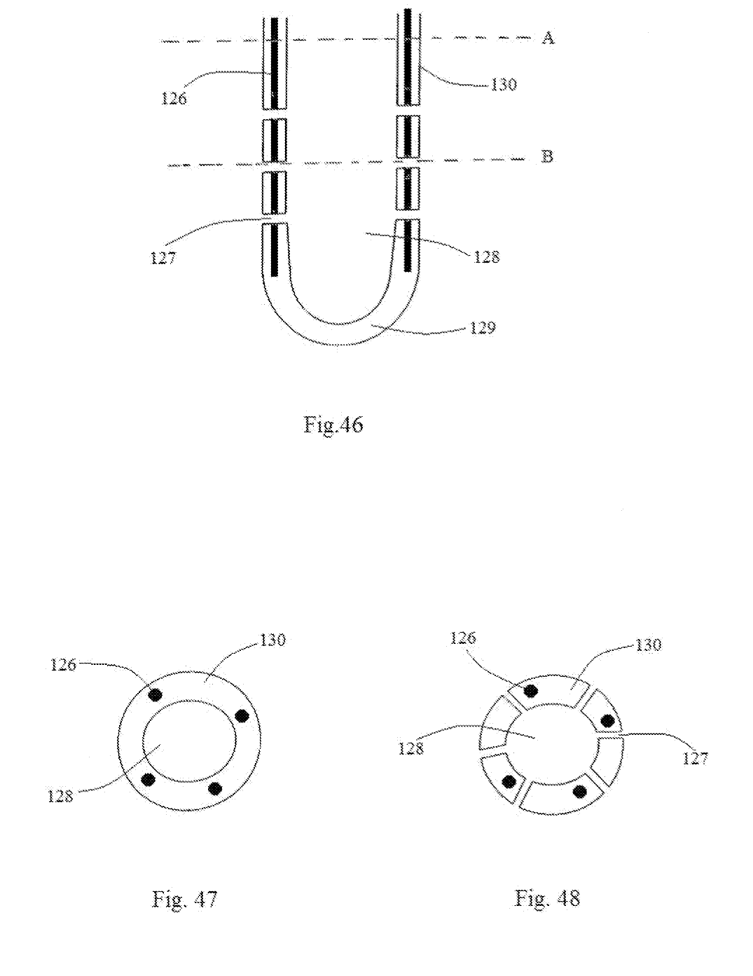

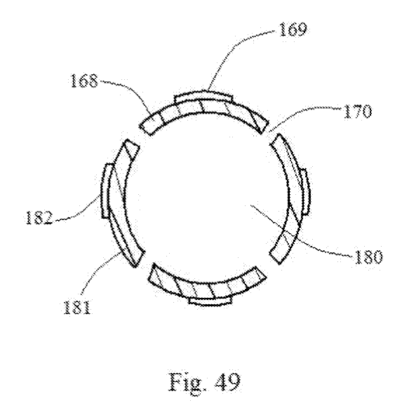

In another embodiment of the ultrasound drain as shown in FIGS. 46-48, the drain wall 130 is embedded with ultrasound conduction wires 126. The distal portion of the drain comprises of ports 127 that connect to the lumen 128. The catheter distal tip 129 is curved allowing for non- traumatic penetration of the brain when placed inside the ventricle with a removable stylet. The ultrasound conduction wires 126 are connected to an external transducer.

In another embodiment of the ultrasonic drain as shown in FIG. 49, the drain comprises of a lumen 180 with drainage holes 170 at the distal drain end. The drain wall 168 and 181 comprise of ultrasound component 169 and 182. The ultrasound component can be either a conductor or transducer. The ultrasonic conductors can also serve as pressure and temperature probes.

The drain wall component can be made from silicone, polyurethane, or any other biocompatible material well known in the art for surgical drain usage. in order to make the drain radio-opaque, the drain wall can either be impregnated with barium or other metallic markers. The drains are usually flexible and in case of a ventriculostomy, a removable stylet is used to create rigidity in the drain for placement through the brain into the ventricle. In other drain embodiments with ultrasound conductors and wires in the wall, the conductor and wires provides a rigid drain component negating the use of a stylet for placement. The wire size can vary from 0.01 mm to 0.5 mm and the number of wires used can vary from 1 to 20. While the above-mentioned size ranges of the drain components reflect many practical embodiments, some alternate embodiments may comprise components outside of the aforementioned ranges.

Drain patency can also be facilitated by the use of negative pressure through the drain lumen. The negative pressure can range from 0 mm Hg to −200 mm Hg. The pressure can be exerted either through a suction bulb connected to the drain, a vacuum regulator, or a gravity drainage system.

While the methodology described herein is specific for central nervous system treatment and prevention of drain obstruction, its use is not limited to this particular pathology. For example, these drains can be used for the treatment of central nervous system hemorrhage for blood clot dissolution and drainage when placed directly into the hemorrhage. These drains can also be used to treat various other central nervous system pathologies. For instance, ultrasonic energy directly transmitted into a brain tumor with the drain system allows tumefaction and dissolution of the tumor cells which can then be drained directly. Similarly the tumefaction process can be facilitated with a direct delivery of a chemotherapeutic agent through the drain.

The invention is thus to cover all modifications, equivalents, and alternatives falling within the spirit and scope of the following claims.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. A central nervous system drain comprising a lumen and a wall; draining fluid through the lumen; and delivering ultrasonic energy through the wall to maintain patency of the lumen.

2. The drain of claim 1, wherein said ultrasound energy is delivered through one or more ultrasound transducers.

3. The drain of claim 1, wherein said ultrasound energy is delivered through one or more ultrasound conductors coupled to a proximal transducer.

4. The drain of claim 1, wherein said ultrasound energy is delivered through an ultrasound conductor with an amplifier.

5. The drain of claim 4, wherein the amplifier is located at the distal end of the drain.

6. The drain of claim 1 comprising one or more ports at the distal end of the wall that communicate from the external environment to the lumen.

7. The drain of claim 1, wherein said draining fluid through the lumen is facilitated by either a suction system or vacuum bulb or gravity drainage bag attached to the distal end of the drain.

8. The drain of claim 1 wherein the said drain also contains probes for central nervous system pressure and temperature monitoring.

9. The drain of claim 1 wherein the said central nervous system comprises of one or more of the following: ventricle, subdural, subarachnoid, epidural, intra-cerebral, intra-thecal, brain, spine, skull, spinal cord.

10. The drain of claim 1 being inserted directly into the central nervous system by one of the following procedures: craniotomy, burr hole, twist drill skull hole, percutaneous skull hole, laminectomy, laminotomy, transforaminal or interlaminar percutaneous spinal placement .

11. A method of treating a hemorrhage in a central nervous system drain comprising the steps of:
inserting a drain into the central nervous system; the drain comprising a lumen and a wall;
delivering ultrasound energy through the drain wall; delivering a hemorrhage lysis agent through the lumen; and draining the hemorrhage through the lumen.

12. The method of claim 11, wherein the hemorrhage lysis agent comprises one or more of the following: i) thrombolytics like streptokinase, urokinase, prourokinase, ancrod, tissue plasminogen activators (alteplase, anistreplase, tenecteplase, reteplase, duteplase), ii) hemolytic agents, iii) antiplatelet agents like GP IIb IIIa, aspirin, ticlopidine, clopidogrel, dipyridamole, iv) anticoagulants like heparin or warfarin, v) fibrinolytic agent like aspirin, vi) thrombolytic agent incorporated into micro-bubbles which can be ultrasonically activated after direct infusion into the blood clot.

13. The method of claim 11, wherein said ultrasound energy is delivered through one or more ultrasound transducers.

14. The method of claim 11, wherein said ultrasound energy is delivered through one or more ultrasound conductors coupled to a proximal transducer.

15. The method of claim 11, wherein said ultrasound delivery means is through one or more ultrasound conductors with a distal amplifier coupled to a proximal transducer.

16. A method of treating a hemorrhage in the central nervous system drain wherein ultrasound energy is used to dissolve the said hemorrhage comprising the steps of: inserting the drain into the central nervous system; the drain comprising two lumens and a wall; delivering ultrasound energy through the drain wall; delivering a hemorrhage lysis agent through the first lumen in the catheter; and draining the hemorrhage through the second lumen.

17. The method of claim 16, wherein the hemorrhage lysis agent comprises one or more of the following: i) thrombolytics like streptokinase, urokinase, prourokinase, ancrod, tissue plasminogen activators (alteplase, anistreplase, tenecteplase, reteplase, duteplase), ii) hemolytic agents, iii) antiplatelet agents like GP IIb IIIa, aspirin, ticlopidine, clopidogrel, dipyridamole, iv) anticoagulants like heparin or warfarin, v) fibrinolytic agent like aspirin, vi) thrombolytic agent incorporated into micro-bubbles which can be ultrasonically activated after direct infusion into the blood clot.

18. The method of claim 16, wherein said ultrasound delivery means is through one or more ultrasound transducers.

19. The method of claim 16, wherein said ultrasound delivery means is through one or more ultrasound conductors coupled to a proximal transducer through wires.

20. The method of claim 16, wherein said ultrasound delivery means is through one or more ultrasound conductors with a distal amplifier coupled to a proximal transducer.
